(19) United States
(12) Patent Application Publication
(10) Pub. No.: US 2014/0018655 A1
(43) Pub. Date: Jan. 16, 2014

(54) BLOOD GLUCOSE METER INTEGRATED WITH A COMPUTING OR COMMUNICATION DEVICE

(71) Applicants: Ramzi ABULHAJ, Charlotte, NC (US); William BAXTER, Holly, MI (US); Amr Yehia Mohamed SHEHAB, Charlotte, NC (US); Moo Nam KO, Charlotte, NC (US); Said MAROUF, Charlotte, NC (US); Elias MAHFOUD, Charlotte, NC (US)

(72) Inventors: Ramzi ABULHAJ, Charlotte, NC (US); William BAXTER, Holly, MI (US); Amr Yehia Mohamed SHEHAB, Charlotte, NC (US); Moo Nam KO, Charlotte, NC (US); Said MAROUF, Charlotte, NC (US); Elias MAHFOUD, Charlotte, NC (US)

(73) Assignee: PRODIGY DIABETES CARE, LLC, Charlotte, NC (US)

(21) Appl. No.: 13/549,636

(22) Filed: Jul. 16, 2012

(51) Int. Cl. A61B 5/145

(52) U.S. Cl. 600/365

(57) ABSTRACT

A blood glucose meter integrated with a computing or communication device that includes a test strip receiving device that is coupled to and powered by the communication port or jack of the computing or communication device and utilizes a blood glucose meter application that resides in the memory and is operated by the microprocessor of the computing or communication device. Advantageously, blood glucose meter data can be transmitted by the computing or communication device to a memory storage “cloud” for later review and/or manipulation by a user, a doctor, and/or the like.

### FIELD OF THE INVENTION

The present invention relates generally to a blood glucose meter integrated with a computing or communication device. More specifically, the present invention relates to a blood glucose meter integrated with a computing or communication device that includes a test strip receiving device that is coupled to and powered by the communication port or jack of the computing or communication device and utilizes a blood glucose meter application that resides in the memory and is operated by the microprocessor of the computing or communication device. Advantageously, blood glucose meter data can be transmitted by the computing or communication device to a memory storage “cloud” for later review and/or manipulation by a user, a doctor, and/or the like.

### BACKGROUND OF THE INVENTION

Unfortunately, diabetes has become a common chronic ailment in the United States and around the World. When insulin in the blood is insufficient, glucose cannot efficiently be converted into energy. The resulting surplus glucose leads to apoplexy and other serious diseases. Thus, a diabetic, even on an insulin treatment regime, must frequently monitor his or her blood glucose level. This is often done using an electronic meter, test strips, and lancets that are used to “prick” a fingertip, such that a blood sample can be collected. Given the fact that many diabetics are older and/or suffer from some degree of visual impairment, electronic meters featuring “voice” or “talking” interfaces and readouts in a variety of languages have become very popular and are widely sold—although such functionality is not universal.

In recent years, the proliferation of mobile communication devices, such as smart phones, tablet computers, and the like has accelerated greatly. Typically, these mobile communication devices include a communication port or jack that accepts headphones or the like and a proprietary or standardized 30-pin universal serial bus (USB) port or the like, used to accept a variety of plug-ins. In fact most plug-ins mate with this 30-pin USB port or the like, at least in part, which is problematic as such connections can be subject to licensing fees and the like. Mobile communication device manufacturers use this 30-pin USB port or the like to limit and control the plug-ins that may be used with their devices.

Thus, what is needed in the art is a blood glucose meter that is coupled to and powered only by the communication port or jack of the computing or communication device and utilizes a blood glucose meter application that resides in the memory and is operated by the microprocessor of the computing or communication device. Preferably, blood glucose meter data could be transmitted by the computing or communication device to a memory storage cloud for later review and/or manipulation by a user, a doctor, and/or the like.

Various attempts have been made to address the above-referenced issues. For example, U.S. Patent Application Publication No. 2010/0249965 (Rao et al.—assigned to AGAMATRIX INC., U.S. patent application Ser. No. 12/749,707, filed Mar. 30, 2010, published Sep. 30, 2010) provides, in one embodiment, a combination comprising a blood glucose meter (BGM) communicatively coupled to a web-enabled portable consumer electronic device (CED) through an audio port of the CED. Data is transferred between the BGM and the CED through the audio port of the CED. The audio port is selected from the group consisting of an audio port pin on a multi-pin connector of the CED and either a headphone audio port of the CED and/or a microphone audio port of the CED. The BGM has a test strip opening sized to receive an electrochemical test strip. Thus, the 30-pin USB port or the like is always utilized. In a second embodiment, the invention provides a method of transferring data between a diagnostic measurement device and a portable consumer electronic device (CED). The method includes a step of communicatively coupling the diagnostic measurement device and the CED through an audio port located on the CED, including the audio port pin on a multi-pin connector of the CED. The method also includes the step of transferring data from the diagnostic measurement device to the CED through the audio port of the consumer electronic device, including the audio port pin on a multi-pin connector of the CED. Thus, again the 30-pin USB port or the like is always utilized.

U.S. Patent Application Publication No. 2010/0279418 (Larson et al., U.S. patent application Ser. No. 12/773,819, filed May 4, 2010, published Nov. 4, 2010) provides a module adaptable to communicate with a suitable handheld device or PDA. Suitable devices include, but are not limited to, the Apple iPhone® or iPod®, Research in Motion Blackberry® smart phones, Motorola Droid smart phones, and Palm Pre smart phones. The module can be used without adding to the cost of the handheld device. This allows direct reimbursement for the replaceable meter module portion if payers choose to limit coverage for the full system, as well as the possibility of reimbursement for the entire system including the handheld device. Other solutions build the cost into the phone, which must be replaced to upgrade or replace the glucose function. Moreover, information from the glucose meter reading can be communicated from the PDA to a remote station for reporting the results. With an iPod-like approach, this could be accomplished without the need for a cellular signal or carrier, as long as a WiFi internet connection is available anywhere in the world. The glucose device described is an attachment module using the standard 30-pin USB connector interface or the like of the handheld device. A single module could be used on multiple handheld devices, saving cost. This flexibility also means that the module could be used with a handheld device, such as an iPod, in the gym, or with a handheld device, such as an iPhone, in the office, etc. Since it is detachable, it does not require extra space or size in the handheld devices itself—it is only attached when a reading is required. It also does not add cost to the handheld device hardware, unlike the integrated units. The functionality of the module could range from a simple electronic interface to the strip (using the handheld device to do all calculations, data processing, display, and communications with health care providers or data services) to an interface plus glucose calculation engine (where the module delivers an answer, and the handheld device provides further data processing, display, and communications with health care providers or data services) to a fully contained meter with a small display, using the handheld device for much richer data processing, display, and communications. An aspect of the disclosure is directed to an apparatus for use to determine blood glucose levels. The apparatus comprises: an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions. Components, such as the logic apparatus and detector can be positioned within a suitable housing or can be configured to be engaged to functionally form a housing. The apparatus is typically handheld. A display screen adapted and configured to display at least one of instructions or measurement results can also be provided. A data processor can be adapted to determine a blood glucose value from a measurement. Another aspect of the disclosure is directed to a method for detecting the blood glucose levels. The method comprises: obtaining a sample from a mammal; applying the sample to a test strip wherein the test strip is inserted into an aperture adapted and configured to receive the strip in an apparatus further comprising a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions; and determining a glucose level from the sample; communicating the glucose level to a handheld apparatus in communication with the blood glucose apparatus. Additional method steps can include, for example, one or more of, instructing a device with mobile communication functionality to contact one or more of an emergency service agency, doctor, and caregiver; displaying results of a the blood glucose measurement; and storing the measurement results on a memory device. Still another aspect of the disclosure is directed to a networked apparatus for determining blood glucose. The networked apparatus comprises: a memory; a processor; a communicator; a display; and an apparatus for detecting a blood glucose level comprising an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions. Still another aspect is directed to communication system. The communication system comprises: an apparatus for detecting blood glucose level comprising an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions; a server computer system; a measurement module on the server computer system for permitting the transmission of a measurement from a system for detecting blood glucose levels over a network; at least one of an API engine connected to at least one of the system for detecting blood glucose levels and the device for detecting blood glucose levels to create an message about the measurement and transmit the message over an API integrated network to a recipient having a predetermined recipient user name, an SMS engine connected to at least one of the system for detecting blood glucose levels and the device for detecting blood glucose levels to create an SMS message about the measurement and transmit the SMS message over a network to a recipient device having a predetermined measurement recipient telephone number, and an email engine connected to at least one of the system for detecting blood glucose levels and the device for detecting blood glucose levels to create an email message about the measurement and transmit the email message over the network to a recipient email having a predetermined recipient email address. Additionally, the system can further comprise a storing module on the server computer system for storing the measurement on the system for detecting blood glucose levels server database. In some configurations at least one of the system for detecting blood glucose levels and the device for detecting blood glucose levels is connectable to the server computer system over at least one of a mobile phone network and an Internet network, and a browser on the measurement recipient electronic device is used to retrieve an interface on the server computer system. Additionally, a plurality of email addresses can be held in a system for detecting blood glucose levels database and fewer than all the email addresses are individually selectable from the diagnostic host computer system, the email message being transmitted to at least one recipient email having at least one selected email address, wherein at least one of the system for detecting blood glucose levels and the device for detecting blood glucose levels is connectable to the server computer system over the Internet, and a browser on the measurement recipient electronic device is used to retrieve an interface on the server computer system. A plurality of user names can be held in the system for detecting blood glucose levels database and fewer than all the user names are individually selectable from the diagnostic host computer system, the message being transmitted to at least one measurement recipient user name via an API. Additionally, measurement recipient electronic device (e.g., smart phone, computer or glucose measurement device) is connectable directly or indirectly to the server computer system over the Internet, and a browser on the measurement recipient electronic device is used to retrieve an interface on the server computer system. Typically, the measurement recipient electronic device is connected to the server computer system over a cellular phone network. In many cases, the measurement recipient electronic device is a mobile device. An interface can also be provided on the server computer system, the interface being retrievable by an application on the mobile device. An SMS message is received by a message application on the mobile device. In some instances, a plurality of SMS messages are received for the measurement, each by a respective message application on a respective recipient mobile device. Typically, at least one SMS engine receives an SMS response over the cellular phone SMS network from the mobile device and stores an SMS response on the server computer system. Additionally, the measurement recipient phone number ID is transmitted with the SMS message to the SMS engine and is used by the server computer system to associate the SMS message with the SMS response. The server computer system can be configured to be connectable over a cellular phone network to receive a response from the measurement recipient mobile device. Additionally, the SMS message can include a URL that is selectable at the measurement recipient mobile device to respond from the measurement recipient mobile device to the server computer system, the server computer system utilizing the URL to associate the response with the SMS message. In some configurations, the system can further comprise, a downloadable application residing on the measurement recipient mobile device, the downloadable application transmitting the response and a measurement recipient phone number ID over the cellular phone network to the server computer system, the server computer system utilizing the measurement recipient phone number ID to associate the response with the SMS message; a transmissions module that transmits the measurement over a network other than the cellular phone SMS network to a measurement recipient user computer system, in parallel with the measurement that is sent over the cellular phone SMS network; and/or a downloadable application residing on the measurement recipient host computer, the downloadable application transmitting a response and a measurement recipient phone number ID over the cellular phone network to the server computer system, the server computer system utilizing the measurement recipient phone number ID to associate the response with the SMS message. Another aspect of the disclosure is directed to a networked apparatus. The networked apparatus comprises: a memory; a processor; a communicator; a display; and an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions. Still another aspect of the disclosure is directed to a communication system. The communication system comprises: an apparatus for detecting blood glucose level comprising an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions; a server computer system; a measurement module on the server computer system for permitting the transmission of a measurement from a system for detecting blood glucose levels over a network; at least one of an API engine connected to at least one of the system for detecting blood glucose levels and the device for detecting blood glucose levels to create an message about the measurement and transmit the message over an API integrated network to a recipient having a predetermined recipient user name, an SMS engine connected to at least one of the system for detecting blood glucose levels and the device for detecting blood glucose levels to create an SMS message about the measurement and transmit the SMS message over a network to a recipient device having a predetermined measurement recipient telephone number, and an email engine connected to at least one of the system for detecting blood glucose levels and the device for detecting blood glucose levels to create an email message about the measurement and transmit the email message over the network to a recipient email having a predetermined recipient email address. A storing module can also be provided on the server computer system for storing the measurement on the system for detecting blood glucose levels server database. In some configurations at least one of the system for detecting blood glucose levels and the device for detecting blood glucose levels is connectable to the server computer system over at least one of a mobile phone network and an Internet network, and a browser on the measurement recipient electronic device is used to retrieve an interface on the server computer system. Additionally, a plurality of email addresses are held in a system for detecting blood glucose levels database and fewer than all the email addresses are individually selectable from the diagnostic host computer system, the email message being transmitted to at least one recipient email having at least one selected email address. In some configurations, at least one of the system for detecting blood glucose levels and the device for detecting blood glucose levels is connectable to the server computer system over the Internet, and a browser on the measurement recipient electronic device is used to retrieve an interface on the server computer system. A plurality of user names can be held in the system for detecting blood glucose levels database and fewer than all the user names are individually selectable from the diagnostic host computer system, the message being transmitted to at least one measurement recipient user name via an API. Moreover, the measurement recipient electronic device is connectable to the server computer system over the Internet, and a browser on the measurement recipient electronic device is used to retrieve an interface on the server computer system. The measurement recipient electronic device can be connected to the server computer system over a cellular phone network, such as where the measurement recipient electronic device is a mobile device. Additionally, an interface on the server computer system, the interface being retrievable by an application on the mobile device. The SMS message can be received by a message application on the mobile device and, in at least some instances, a plurality of SMS messages are received for the measurement, each by a respective message application on a respective recipient mobile device. At least one SMS engine can be configured to receive an SMS response over the cellular phone SMS network from the mobile device and stores an SMS response on the server computer system. A measurement recipient phone number ID is transmitted with the SMS message to the SMS engine and is used by the server computer system to associate the SMS message with the SMS response. A server computer system is connectable over a cellular phone network to receive a response from the measurement recipient mobile device. The SMS message can includes, for example, a URL that is selectable at the measurement recipient mobile device to respond from the measurement recipient mobile device to the server computer system, the server computer system utilizing the URL to associate the response with the SMS message. The system can further include a downloadable application residing on the measurement recipient mobile device, the downloadable application transmitting the response and a measurement recipient phone number ID over the cellular phone network to the server computer system, the server computer system utilizing the measurement recipient phone number ID to associate the response with the SMS message; a transmissions module that transmits the measurement over a network other than the cellular phone SMS network to a measurement recipient user computer system, in parallel with the measurement that is sent over the cellular phone SMS network; a downloadable application residing on the measurement recipient host computer, the downloadable application transmitting a response and a measurement recipient phone number ID over the cellular phone network to the server computer system, the server computer system utilizing the measurement recipient phone number ID to associate the response with the SMS message. Thus, again the 30-pin USB port or the like is always utilized.

In an unrelated field, U.S. Patent Application Publication No. 2012/0126020 (Babu et al., U.S. patent application Ser. No. 13/298,484, filed Nov. 17, 2011, published May 24, 2012) provides a read head configured to be coupled to a mobile device. The read head has a slot for swiping a magnetic stripe of a card. The read head reads data on the magnetic stripe and produces a raw magnetic signal indicative of data stored on the magnetic stripe. Device electronics are provided with an analog front-end and a microcontroller. The analog to digital front end is coupled to a processing element in the microcontroller. The analog to digital front end receives a raw magnetic head signal and converts it into a processed digital signal that the microcontroller can interpret. The microcontroller produces a signal. An output jack is adapted to be inserted in a port of the mobile device and deliver an output jack signal to the mobile device.

Thus, what is still needed in the art is a blood glucose meter that is coupled to and powered only by the communication port or jack of the computing or communication device and utilizes a blood glucose meter application that resides in the memory and is operated by the microprocessor of the computing or communication device. Preferably, blood glucose meter data could be transmitted by the computing or communication device to a memory storage cloud for later review and/or manipulation by a user, a doctor, and/or the like.

### BRIEF SUMMARY OF THE INVENTION

In various exemplary embodiments, the present invention provides a blood glucose meter integrated with a computing or communication device, such as a smart phone, tablet computer, or the like, that includes a test strip receiving device that is coupled to and powered by the communication port or jack of the computing or communication device and utilizes a blood glucose meter application that resides in the memory and is operated by the microprocessor of the computing or communication device. Advantageously, blood glucose meter data can be transmitted by the computing or communication device to a memory storage cloud for later review and/or manipulation by a user, a doctor, and/or the like.

In one exemplary embodiment, the present invention provides a blood glucose meter system, comprising: a test strip receiving device operable for selectively receiving and testing a blood glucose test strip and comprising a communication port connector configured to selectively couple the test strip receiving device to a communication port of a computing or communication device; wherein the communication port connector is operable for delivering power from the computing or communication device to the test strip receiving device when coupled; and wherein the communication port connector is operable for transferring data between the test strip receiving device and the computing or communication device when coupled. The blood glucose meter system also comprises an application disposed within a memory and executed by a microprocessor of the computing or communication device for interacting with and controlling the test strip receiving device. The application is selectively downloaded from a provider server and/or external media. A user interacts with the application through a display of the computing or communication device. The blood glucose meter system further comprises a provider server and/or external media coupled to the computing or communication device through a network. The data is transferred from the test strip receiving device to the provider server and/or external media through the network by the computing or communication device. The data is retrievable from the provider server and/or external media by one or more of a verified user, a verified patient, a verified doctor, and a verified provider.

In another exemplary embodiment, the present invention provides a blood glucose meter method, comprising: providing a test strip receiving device operable for selectively receiving and testing a blood glucose test strip and comprising a communication port connector configured to selectively couple the test strip receiving device to a communication port of a computing or communication device; wherein the communication port connector is operable for delivering power from the computing or communication device to the test strip receiving device when coupled; and wherein the communication port connector is operable for transferring data between the test strip receiving device and the computing or communication device when coupled. The blood glucose meter method also comprises providing an application disposed within a memory and executed by a microprocessor of the computing or communication device for interacting with and controlling the test strip receiving device. The application is selectively downloaded from a provider server and/or external media. A user interacts with the application through a display/interface of the computing or communication device. The blood glucose meter method further comprises providing a provider server and/or external media coupled to the computing or communication device through a network. The data is transferred from the test strip receiving device to the provider server and/or external media through the network by the computing or communication device. The data is retrievable from the provider server and/or external media by one or more of a verified user, a verified patient, a verified doctor, and a verified provider.

### BRIEF DESCRIPTION OF THE DRAWINGS

The present invention is illustrated and described herein with reference to the various drawings, in which like reference numbers are used to denote like device components/method steps, as appropriate, and in which:

FIG. 1 is a schematic diagram illustrating one exemplary embodiment of the test strip receiving device of the present invention coupled to a computing or communication device; and

FIG. 2 is a schematic diagram illustrating one exemplary embodiment of the mobile blood glucose meter system of the present invention.

### DETAILED DESCRIPTION OF THE INVENTION

Again, in various exemplary embodiments, the present invention provides a blood glucose meter integrated with a computing or communication device (i.e. a computer or mobile communication device), such as a desktop, a laptop, a smart phone, a tablet computer, a handheld computer, a portable reader, or the like, that includes a test strip receiving device that is coupled to and powered by the communication port (i.e. the audio port) or jack of the computing or communication device and utilizes a blood glucose meter application that resides in the memory and is operated by the microprocessor of the computing or communication device. Advantageously, blood glucose meter data can be transmitted by the computing or communication device to a memory storage cloud for later review and/or manipulation by a user, a doctor, and/or the like.

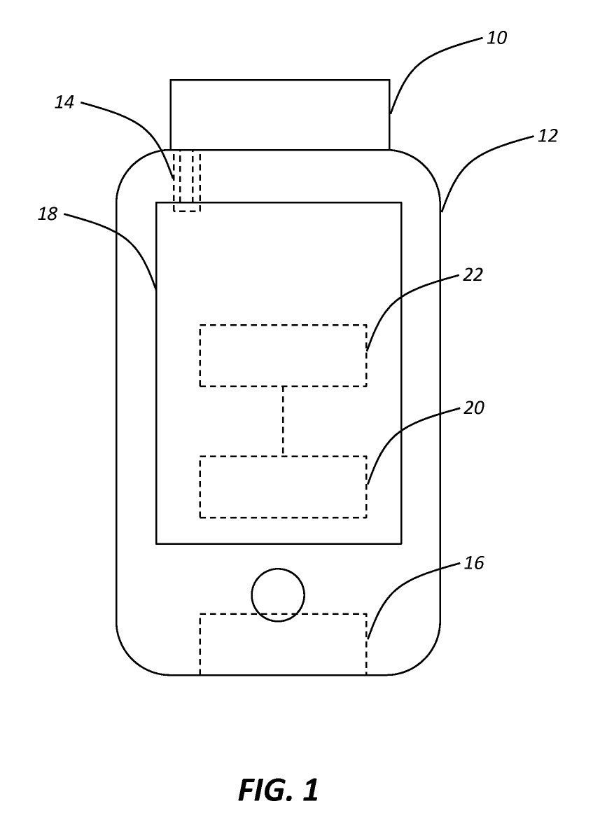

Referring now specifically to FIG. 1, in one exemplary embodiment, the present invention provides a test strip receiving device 10, well known to those of ordinary skill in the art for receiving and electrically testing a blood sample disposed on a test strip in order to determine the glucose content of the blood sample, coupled to a computing or communication device 12, such as a smart phone, tablet computer, or the like. Specifically, the test strip receiving device 10 is coupled to the communication port or jack 14 of the computing or communication device 12, as opposed to the 30-pin USB port or the like 16, as is typically provided. The test strip receiving device 10 derives power from and shares data via the communication port or jack 14, and may include any desired power indicators or other user interfaces and/or displays. Accordingly, the test strip receiving device 10 does not have to include its own internal power supply and may be a relatively simple and inexpensive device.

The communication port or jack 14, typically used to receive a headset or the like, can be used to parasitically power external peripherals and transfer data to and from them—using analog, digital, or serial signaling. The typical connections associated with the communication port or jack 14 include a left earphone connection (at the tip), a right earphone connection (at the first ring), a common/ground connection (at the second ring), and a microphone connection (at the sleeve), although other connections can be utilized. It has been found that the measured impedance between the earphones and the common is about 33Ω, while the measured impedance between the microphone and the common is about 640Ω, for example (see Kuo et al., “Hijacking Power and Bandwidth From the Mobile Phone's Audio Interface,” ACM DEV '10, Dec. 17-18, 2010, London, United Kingdom). Thus, it has been found that maximum power transfer occurs at about 240 mVrms when delivering about 66 mArms, with a load impedance of about 3.6Ω, for example. Thus, there is sufficient power to feed the test strip receiving device 10 of the present invention, and data may be shared thereby and therewith via the communication port or jack 14. One manner in which this may be accomplished is by having the test strip receiving device send a sine wave that is converted to a square wave, thereby providing 3+ V DC, for example.

Internally, a blood glucose meter application is stored in the memory 20 of the computing or communication device 12, and the microprocessor 22 of the computing or communication device 12 is used to run the blood glucose meter application and interface with the test strip receiving device 10. Thus, the display/interface 18 of the computing or communication device 12 is used to provide instructions and display results to the user in the glucose testing of a blood sample. The display/interface 18 of the computing or communication device 12 can also be used by the user to review historical results stored in the memory 20 of the computing or communication device 12, direct results to be sent to one or more external storage repositories (as described in greater detail herein below), etc. Advantageously, the blood glucose meter application can be controlled by and obtained from a manufacturer or distributor of the test strip receiving device 10, and the manufacturer or distributor of the test strip receiving device 10 can control and maintain the one or more external storage repositories—in a memory storage “cloud” for later review and/or manipulation by the user, a doctor, and/or the like, for example. The blood glucose meter application can be a free application, with limited historical storage capabilities or, alternatively, the blood glucose meter application can be a pay application, with robust historical storage capabilities, for example.

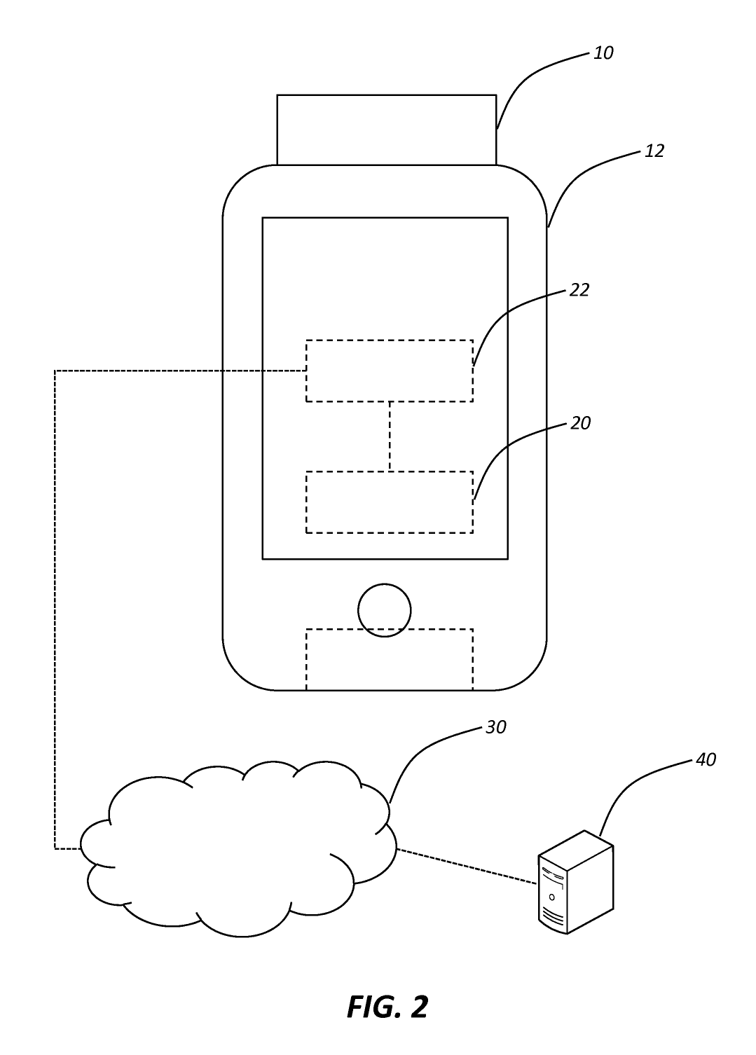

Referring now specifically to FIG. 2, in one exemplary embodiment, the present invention provides a system and method by which the blood glucose readings of a patient are taken using the test strip receiving device 10, computing or communication device 12, and blood glucose meter application of the present invention and transferred to a remote storage repository for later sorting, reading, and/or review by the patient, a doctor, and/or the like. Specifically, data collected using the test strip receiving device 10, computing or communication device 12, and blood glucose meter application is transferred to a free or subscriber network 30 via the long-range or short-range wireless capabilities of the computing or communication device 12, just as other data is transferred. The data is then stored in a provider server 40 and/or external media, such as a provider web server accessible through a website and the Internet. By accessing the data, a patient can review historical blood glucose test results, as can a doctor (and the doctor can bill the patient for such review). Further, a blood glucose meter supplier can review the historical blood glucose test results to determine when the patient need more test strips, for example. Preferably, the data is secured by an appropriate access code and/or other security measures. This system and method by which the blood glucose readings of the patient are taken using the test strip receiving device 10, computing or communication device 12, and blood glucose meter application of the present invention can be integrated with any existing electronic medical records (EMR) system currently in existence.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A blood glucose meter system, comprising:
a test strip receiving device operable for selectively receiving and testing a blood glucose test strip and comprising a communication port connector configured to selectively couple the test strip receiving device to a communication port of a computing or communication device;
wherein the communication port connector is operable for delivering power from the computing or communication device to the test strip receiving device when coupled; and
wherein the communication port connector is operable for transferring data between the test strip receiving device and the computing or communication device when coupled.

2. The blood glucose meter system of claim 1, further comprising an application disposed within a memory and executed by a microprocessor of the computing or communication device for interacting with and controlling the test strip receiving device.

3. The blood glucose meter system of claim 2, wherein the application is selectively downloaded from a provider server and/or external media.

4. The blood glucose meter system of claim 2, wherein a user interacts with the application through a display/interface of the computing or communication device.

5. The blood glucose meter system of claim 1, further comprising a provider server and/or external media coupled to the computing or communication device through a network.

6. The blood glucose meter system of claim 4, wherein the data is transferred from the test strip receiving device to the provider server and/or external media through the network by the computing or communication device.

7. The blood glucose meter system of claim 5, wherein the data is retrievable from the provider server and/or external media by one or more of a verified user, a verified patient, a verified doctor, and a verified provider.

8. A blood glucose meter method, comprising:
providing a test strip receiving device operable for selectively receiving and testing a blood glucose test strip and comprising a communication port connector configured to selectively couple the test strip receiving device to a communication port of a computing or communication device;
wherein the communication port connector is operable for delivering power from the computing or communication device to the test strip receiving device when coupled; and
wherein the communication port connector is operable for transferring data between the test strip receiving device and the computing or communication device when coupled.

9. The blood glucose meter method of claim 8, further comprising providing an application disposed within a memory and executed by a microprocessor of the computing or communication device for interacting with and controlling the test strip receiving device.

10. The blood glucose meter method of claim 9, wherein the application is selectively downloaded from a provider server and/or external media.

11. The blood glucose meter method of claim 9, wherein a user interacts with the application through a display/interface of the computing or communication device.

12. The blood glucose meter method of claim 8, further comprising providing a provider server and/or external media coupled to the computing or communication device through a network.

13. The blood glucose meter method of claim 12, wherein the data is transferred from the test strip receiving device to the provider server and/or external media through the network by the computing or communication device.

14. The blood glucose meter method of claim 13, wherein the data is retrievable from the provider server and/or external media by one or more of a verified user, a verified patient, a verified doctor, and a verified provider.
